SEQUENCE LISTING (36 sequences)

SEQ ID NO: 1 - DNA, 639 bp, Hepatitis B virus
  CDS (1)..(639)
atgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 2 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 3 - protein, 4 aa, Hepatitis B virus
ESQC

SEQ ID NO: 4 - protein, 5 aa, Hepatitis B virus
RESQC

SEQ ID NO: 5 - protein, 6 aa, Hepatitis B virus
SRESQC

SEQ ID NO: 6 - protein, 7 aa, Hepatitis B virus
QSRESQC

SEQ ID NO: 7 - protein, 8 aa, Hepatitis B virus
SQSRESQC

SEQ ID NO: 8 - DNA, 20 bp, Artificial sequence
  Primer
ctgcactcaggcaagccatt

SEQ ID NO: 9 - DNA, 20 bp, Artificial sequence
  Primer
gccgaggcaggtcccctaga

SEQ ID NO: 10 - DNA, 20 bp, Artificial sequence
  Primer
gaaaatcttctcggatccgc

SEQ ID NO: 11 - DNA, 20 bp, Artificial sequence
  Primer
agagatctccatggattcag

SEQ ID NO: 12 - DNA, 21 bp, Artificial sequence
  Primer
gtggctttggggccatggaca

SEQ ID NO: 13 - DNA, 27 bp, Artificial sequence
  Primer
aggactagtgcctcggccccgtcgtct

SEQ ID NO: 14 - DNA, 27 bp, Artificial sequence
  Primer
agaactagtcaatctagggaatctcaa

SEQ ID NO: 15 - DNA, 24 bp, Artificial sequence
  Primer
tcttctaacactagtagtttccgg

SEQ ID NO: 16 - DNA, 24 bp, Artificial sequence
  Primer
cagctagcgcaatttccatccgta

SEQ ID NO: 17 - DNA, 24 bp, Artificial sequence
  Primer
gtttgtgctagcattccaggaaca

SEQ ID NO: 18 - DNA, 24 bp, Artificial sequence
  Primer
ccataggttgctagcgaaagccca

SEQ ID NO: 19 - DNA, 24 bp, Artificial sequence
  Primer
ttgctagcgttcagcgcagggtcc

SEQ ID NO: 20 - DNA, 24 bp, Artificial sequence
  Primer
gtgagagctagctatttccctgct

SEQ ID NO: 21 - protein, 63 aa, Artificial sequence
  Hepatitis B core derivative
PKRSISKRSISDPAFRANTANPDWDFNPNKDTWPDANKVGAGAFGLGFTPPHGGLLGWSPQSE

SEQ ID NO: 22 - protein, 64 aa, Artificial sequence
  Hepatitis B core derivative
PKRSIQAMQWNSTTFHQTLQDPRVRGLYFPAGGSSSGTVNPVPTTVSPISSIFSRIGDPALKSE

SEQ ID NO: 23 - protein, 68 aa, Artificial sequence
  Hepatitis B core derivative
PKRSISKRSISDPAFRANTANPDWDFNPNKDTWPDANKVGAGAFGLGFTPPHGGLLGWSPHDIGDYCC

SEQ ID NO: 24 - protein, 67 aa, Artificial sequence
  Hepatitis B core derivative
PKRSIQAMQWNSTTFHQTLQDPRVRGLYFPAGGSSSGTVNPVPTTVSPISSIFSRIGDPALNGDYCC

SEQ ID NO: 25 - protein, 101 aa, Artificial sequence
  Hepatitis B core derivative
PNSDTGHSSQVSQNYPIVQNIQGQMVHQAISPRTLNAWVKVVEEKAFSPEVIPMFSALSEGATPQDLNTMLNTVGGHQAAMQMLKETINEEAAAGMQASLA

SEQ ID NO: 26 - protein, 158 aa, Hepatitis B virus
MDSDNPASTTNKDKDPRALGWLWGMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRP

SEQ ID NO: 27 - protein, 51 aa, Hepatitis B virus
PNAPILSTLPETTVVRRRGRGRSPRRRTPSPRRRRSQSPRRRRSQSRESQC

SEQ ID NO: 28 - protein, 28 aa, Hepatitis B virus
PNAPILSTLPETTVVRRRGTSQSRESQC

SEQ ID NO: 29 - protein, 20 aa, Hepatitis B virus
PNAPILSTLPETSQSRESQC

SEQ ID NO: 30 - DNA, 640 bp, Hepatitis B virus
agagatctccatggattcagataatccagctagtactaccaacaaagataaggatccgcgcgcccttgggtggctttggggcatggacattgacccttataaagaatttggagctactgtggagttactctcgtttttgccttctgacttctttccttccgtcagagatcttctagacaccgcctcagctctgtatcgagaagccttagagtctcctgagcattgctcacctcaccatactgcactcaggcaagccattctctgctggggggaattgatgactctagctacctgggtgggtaataatttggaagatccagctagcagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacggggccgaggcaggtcccctagaagaagaactccctcgcctcgcagacgcagatctcaatcgccgcgtcgcagaagatctcaatctagggaatctcaatgttag

SEQ ID NO: 31 - DNA, 108 bp, Hepatitis B virus
  CDS (1)..(108)
ctgtatttccctgctggtggctccagttcaggaacagtaaaccctgttctgactactgcctctcccttatcgtcaatcttctcgaggattggggaccctgcgctgaac

SEQ ID NO: 32 - protein, 36 aa, Hepatitis B virus
LYFPAGGSSSGTVNPVLTTASPLSSIFSRIGDPALN

SEQ ID NO: 33 - DNA, 114 bp, Hepatitis B virus
  CDS (1)..(114)
attccaggaacaacaacaaccagtacgggaccatgcaaaacctgcacgactcctgctcaaggcaactctatgtttccctcatgttgctgtacaaaaccttcggatggaaattgc

SEQ ID NO: 34 - protein, 38 aa, Hepatitis B virus
IPGTTTTSTGPCKTCTTPAQGNSMFPSCCCTKPSDGNC

SEQ ID NO: 35 - DNA, 144 bp, Hepatitis B virus
  CDS (1)..(144)
attccaggaacaacaacaaccagtacgggaccatgcaaaacctgcacgactcctgctcaaggcaactctatgtttccctcatgttgctgtacaaaaccttcggatggaaattgcacctgtattcccatcccatcgtcttgggct

SEQ ID NO: 36 - protein, 48 aa, Hepatitis B virus
IPGTTTTSTGPCKTCTTPAQGNSMFPSCCCTKPSDGNCTCIPIPSSWA

(19) United States
(12) Patent Application Publication
(10) Pub. No.: US 2004/0054139 A1
(43) Pub. Date: Mar. 18, 2004

(54) Modification of hepatitis b core antigen

(76) Inventors: Mark Page, Derby (GB); Jin-Li Li, Beckenham (GB); Paul Pumpens, Riga Latvia (GB); Galina Borisova, Riga Latvia (GB)

Correspondence Address: NIXON & VANDERHYE, PC, 1100 N GLEBE ROAD, 8TH FLOOR, ARLINGTON, VA, 22201-4714, US

(21) Appl. No.: 10/312,045

(22) PCT Filed: Jun. 22, 2001
(86) PCT No.: PCT/GB01/02817

(30) Foreign Application Priority Data
Jun. 22, 2000 (GB) 0015308.0
Oct. 6, 2000 (GB) 0024544.9

(51) Int. Cl. C07K 14/74

(52) U.S. Cl. 530/350

(57) ABSTRACT

A protein is provided comprising hepatitis B core antigen (HBcAg) wherein one or more of the four arginine repeats has been deleted, said protein comprising the C-terminal cysteine of HBcAg. The deleted region may be replaced by an epitope from a protein other than HBcAg, in which case the HBcAg acts as a carrier to present the epitope to the immune system. The chimeric protein is useful in prophylactic and therapeutic vaccination of a host, for example against hepatitis B virus.

The invention relates to modified forms of the core antigen of hepatitis B virus (HBV) and to prophylactic and therapeutic vaccines containing the modified antigen.

### BACKGROUND TO THE INVENTION

HBV remains a major healthcare problem throughout both the developed and developing world. Infection with the virus can result in an acute or chronic disease which in a proportion of cases may lead to hepatocellular carcinoma and death. The virus is double shelled, and its DNA is protected inside a protein structure called the core antigen (HBcAg). The core is surrounded by the envelope protein known as the surface or S antigen (HBsAg).

HBcAg is an unusual antigen which can be used as a delivery vehicle for specific peptides to the immune system. The antigen has been used to present T-helper, B and cytotoxic lymphocyte (CTL) epitopes from a variety of viral and bacterial pathogens, including epitopes from the surface antigen of HBV, envelope proteins from hepatitis A and antigens from hepatitis C virus. For a review see Ulrich et al (1998) Advances in Virus Research 50 141-182.

HBcAg is an excellent vehicle for the presentation of epitopes due to the molecular structure of the protein, which self-assembles into particles. Each particle is generated from either 180 or 240 copies of a monomeric polypeptide. The polypeptide has 183 or 185 amino acids (aa) depending on the subtype of HBV. The monomer, on reaching an appropriate concentration inside the host cell, forms a particle of approximately 27 nm in diameter. Structural studies have shown that amino acids within the region from residues 68 to 90 form a spiked structure on the surface of the particle which is known as the e1 loop. Two monomers joined by disulphide bonds link to form a dimer spike, the most exposed amino acid being at position 80 (at the centre of the e1 loop).

EP-A-421635 (The Wellcome Foundation Limited) describes modification of the HBV core gene to allow insertion of foreign epitopes into the e1 loop without altering the potential of the protein to from particles. Insertion at this site allows maximum exposure of the inserted epitope on the tip of each spike created by dimers of the protein. As there are approximately 180 (or 240) copies of each monomer per particle, each particle is able to present 180 (or 240) copies of the epitope of interest.

Thus, HBcAg can be used to generate hybrid particles to be used as prophylactic and therapeutic vaccines against infectious diseases. However, initial work has identified a high nucleic acid impurity profile due to the inherent nature of the core protein to bind nucleic acid. The binding of nucleic acid is known to be associated with four arginine repeats found at the C-terminus of the protein. Removal of these repeats using genetic tools has been shown to be feasible and results in the production of particles which do not encapsidate nucleic acid. However, removal of this region appears to reduce the inherent stability of the particle structure.

### SUMMARY OF THE INVENTION

In order to maintain particle stability, whilst overcoming the problem of nucleic acid impurity, the inventors have devised an alternative and novel strategy. The strategy involves generating a clone in which one or more of the arginine repeats of HBcAg is removed but in which the C-terminal cysteine is retained. The removal of the arginine repeats reduces binding of nucleic acid, whilst retention of the C-terminal cysteine allows the formation of a disulphide bond which in the native structure is important for the formation of a stable particle. The deleted repeat(s) may be replaced with sequences encoding T-helper, B or CTL epitopes from bacterial or viral pathogens, parasites, allergens or cancer associated antigens. This is made possible by insertion of a suitable cloning site in place of the deleted region.

Thus, the invention provides a protein comprising HBcAg wherein one or more of the four arginine repeats is absent and a C-terminal cysteine residue is present. An epitope from a protein other than BHcAg may be present in place of the absent arginine repeat(s). The protein may be incorporated into a pharmaceutical composition for prophylactic or therapeutic vaccination, for example against HBV.

The protein of the invention may comprise a second epitope from a protein other than HBcAg, and the second epitope may be in the e1 loop of HBcAg. By placing a T-helper epitope in the C-terminus and a B-cell epitope in the e1 loop, it is possible to enhance the response to the B-cell epitope through intrastructural T-cell help. In addition, the strategy can be used to double the number of a particular epitope on each particle, by cloning the same sequence into both the e1 loop and the C-terminal region.

### BRIEF DESCRIPTION OF THE DRAWINGS

FIG. 1: Amino acid sequence of hepatitis B core using the single letter code. The C-terminal sequence (aa135-185) is highlighted to detail the deletion strategy. The 4 arginine (R) repeats are emboldened and underlined for emphasis. Three or four arginine repeat regions are underlined from aa154-178 or aa146-178 respectively. Deletion of the underlined regions with insertion of the SpeI restriction site generates constructs encoded by plasmids pTCR₁₅₄ and pTCR₁₄₆ respectively. pTCR₁₅₄ retains the N-terminal arginine repeat, and pTCR₁₄₆ has all 4 arginine repeats deleted.

FIG. 2: DNA sequence coding for HBcAg and location and orientation of oligonucleotide primers used for PCR. The position of the SpeI restriction site is given for oligos MGR371, MGR369 and MGR370 (see Table 1).

FIG. 3: DNA and amino acid sequences of pre-S2 and S epitopes inserted into core.

FIG. 3A shows the sequence of aa20-55 of the pre-S2 region of the HBV ayw subtype.

FIG. 3B shows the sequence of aa110-147 of the S antigen of the adw subtype. FIG. 3C shows the sequence of aa110-157 of the S antigen of the adw subtype.

FIG. 4: Agarose gel electroporesis of inverse PCR fragments. Lanes 1, 2, 3 and 4=fragments for pTCR₁₄₆, pTCR₁₅₄, pTCSR₁₄₆ and pTCSR₁₅₄ respectively. Lane 5=size markers. All fragments are of about 5 kb as expected.

FIG. 5: Immunoblot analysis of expression of core protein in lysates of E.coli bacteria transformed with 3′ replacement plasmid constructs. All samples express an anti-core antibody reactive protein of various relative molecular weights depending on presence or absence of replacement sequences and size of replacement. Sample order:

Lane 1=pTCR₁₄₆ E.coli HB101

Lane 2=pTCR₁₄₆/S110-157 E.coli HB101

Lane 3=pTCR₁₄₆/S2-2 E.coli HB101

Lane 4=pTCR₁₅₄ E.coli HB101

Lane 5=pTCR₁₅₄/S110-147 E. coli HB101

Lane 6=pTCR₁₅₄/S110-157 E.coli HB101

Lane 7=pTCR₁₅₄/S2-2 E.coli HB101

Lane 8=pTCSR₁₄₆ E.coli HB101

Lane 9=pTCSR₁₄₆/S110-157 E.coli HB101

Lane 10=pTCSR₁₄₆/S2-2 E.coli HB101.

FIG. 6: Immunoblot analysis of expression of S sequence in lysates of bacteria transformed with 3′ replacement plasmid constructs. Constructs incorporating the S sequences (lanes 2, 4, 5 and 7) are anti-S antibody reactive. Sample order:

Lane 1=pTCR₁₄₆ E.coli HB101

Lane 2=pTCR₁₄₆/S110-157 E.coli HB101

Lane 3=pTCR₁₅₄ E.coli HB101

Lane 4=pTCR₁₅₄/S110-147 E.coli HB101

Lane 5=pTCR₁₅₄/S110-157 E.coli HB101

Lane 6=pTCSR₁₄₆ E.coli HB101

Lane 7=pTCSR₁₄₆/S11-157 E.coli HB101

Lane 8=Pre-stain marker (Novex).

FIG. 7: Immunoblot analysis of expression of pre-S2 sequence in lysates of bacteria transformed with 3′ replacement plasmid constructs. Constructs incorporating the pre-S2 sequences (lanes 2, 4 and 6) are pre-S2 antibody reactive. Sample order:

Lane 1=pTCR₁₄₆ E.coli HB101

Lane 2=pTCR₁₄₆/S2-2 E.coli HB101

Lane 3=pTCR₁₅₄ E.coli HB101

Lane 4=pTCR₁₅₄/S2-2 E.coli HB101

Lane 5=pTCSR₁₄₆ E.coli HB101

Lane 6=pTCSR₁₄₆/S2-2 E.coli HB101

Lane 7=Pre-stain marker (Novex).

FIG. 8: shows averaged anti-HBc responses in mice immunised with various constructs described in the Examples. The titers were calculated as the negative logarithms of the EC50 (effective concentration, 50%) serum dilution on the basis of sigmoidal dose-response curves.

### DETAILED DESCRIPTION OF THE INVENTION

The Modifications to the HBcAg Sequence

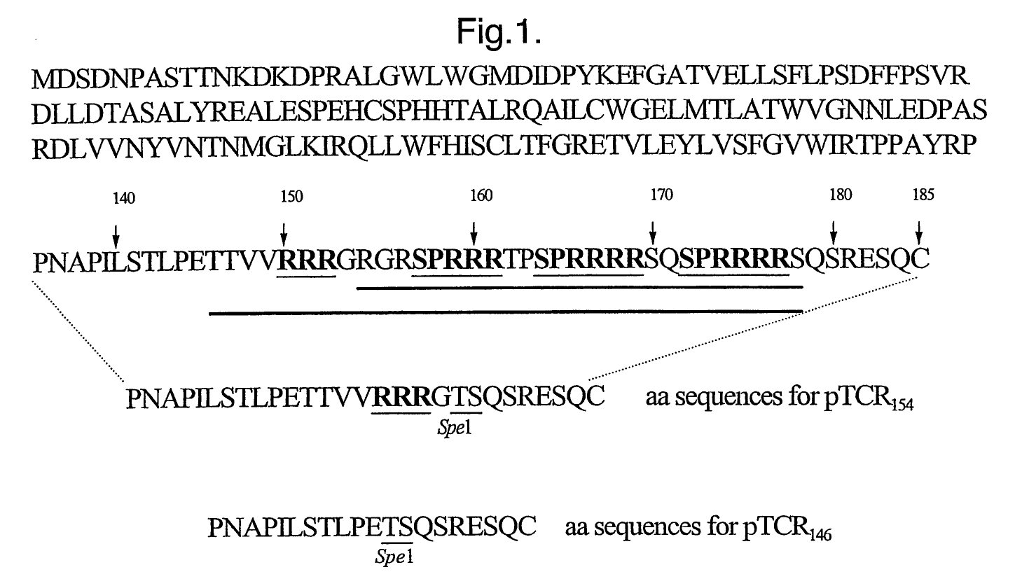

As mentioned above, HBcAg is a protein of 183 or 185 amino acids depending on the sub-type of HBV. The extra two amino acids in the 185 form of the protein are located between the first and the second arginine repeats. The sequence of a 185 amino acid form of the protein with a pre-sequence is shown in FIG. 1. In FIG. 1, the mature HBcAg sequence runs from the Met residue at position 25 to the Cys residue at the extreme C-terminus, with the sequence from residues 1 to 24 being the pre-sequence. The four arginine repeats are located at the following positions:

| | | Position in mature |
|---|---|---|
| | Position in mature | 185 aa sequence |
| | 183 aa sequence | (see FIG. 1) |
| furst repeat | 150-152 | 150-152 |
| second repeat | 157-159 | 159-161 |
| third repeat | 164-167 | 166-169 |
| fourth repeat | 172-175 | 174-177 |

One or more of the arginine repeats is deleted in the protein of the invention. Thus, it is ossible to delete one, two, three or all four of the repeats and to delete the first repeat, the second repeat, the third repeat and/or the fourth repeat. Any combination of the four repeats may be deleted. The first repeat is primarily responsible for RNA binding and the second, third and fourth repeats are primarily responsible for DNA binding, and in a referred embodiment the first repeat is retained and the second to fourth repeats are deleted in order to specifically reduce DNA binding.

A sequence lying between residues 145 and 182 of HBcAg is generally absent in the proteins of the invention, and preferably a sequence lying between residues 150 and 177 is absent. The deleted sequence may comprise the whole of the sequence from residue 145 to residue 182 (or from residue 150 to residue 177) or may comprise only apart of the sequence between those residues. Equally, the deleted sequence may extend on either side of those residues. As used herein, expressions such as “a sequence lying between residues x and y is absent” mean that the sequence which is absent may include residues x and y. Removal of sequence upstream of residue 145 may interfere with the particle-forming ability of the protein and is therefore generally not recommended. In 185 aa forms of HBcAg the deleted sequence may end at residue 184, and in 183 aa forms it may end at residue 182.

The C-terminal cysteine residue in the protein of the invention is typically the natural residue from the C-terminus of HBcAg and is typically preceded by the sequence immediately upstream of the residue in HBcAg. The preceding HBcAg sequence may comprise from 1 to 7 residues, i.e. 1, 2, 3, 4, 5, 6 or 7 residues. Thus, the C-terminus of the protein of the invention may have the sequence Gln Cys, Ser Gln Cys, Glu Ser Gln Cys, Arg Glu Ser Gln Cys, Ser Arg Glu Ser Gln Cys, Gln Ser Arg Glu Ser Gln Cys or Se, Gln Ser Arg Glu Ser Gln Cys. However, the Cys residue may not be the one from HBcAg; in this case, a protein according to the invention may be constructed by truncating the HBcAg sequence and replacing the truncated sequence with another sequence including a Cys residue and optionally an epitope from a protein other than HBcAg. The Cys residue is typically located at the extreme C-terminal end of the protein of the invention but it may be a number of amino acid residues from the extreme C-terminal end. For example, it may be from 1 to 20, from 1 to 10 or from 1 to 5 residues from the C-terminus. In any event, the Cys residue must be able to form a disulphide bond.

The protein of the invention typically comprises the following elements linked in an N-terminal to C-terminal direction:

(i) an N-terminal part of HBcAg which mediates the formation of particles, for example residues 1 to 144 (or 1 to 146 or 1 to 154), and

(ii) a C-terminal part of HBcAg comprising the C-terminal cysteine; wherein at least a part of the sequence of HBcAg from between said N-terminal part and said C-terminal part comprising one or more of the arginine repeats is absent.

Where the protein also comprises an epitope from a protein other than HBcAg in place of the absent arginine repeat(s), the protein typically comprises the following elements linked in an N- to C-terninal direction:

(i) an N-terminal part of HBcAg which mediates the formation of particles, for example residues 1 to 144 (or 1 to 146 or 1 to 154),

(ii) an epitope from a protein other than HBcAg, and

(iii) a C-terminal part of HBcAg comprising the C-terminal cysteine;

wherein at least part of the sequence of HBcAg between said N-terminal part and said C-terminal part comprising one or more of the arginine repeats is absent and is replaced by said epitope.

Where the protein comprises an epitope from a protein other than HBCAg in the e1 loop, the protein typically comprises the following elements linked in an N- to C-terminal direction:

(i) an N-terminal part of the HBcAg sequence comprising e.g. residues 1 to 67 (or 1 to 74 or 1 to 79),

(ii) an epitope from a protein other than HBcAg,

(iii) a second part of the HBcAg sequence comprising e.g. residues 91 to 144 (or 91 to 146, 91 to 154, 86 to 144, 86 to 146, 86 to 154, 80 to 144, 80 to 146 or 80 to 154); and

(iv) a third part of the HBcAg sequence comprising the C-terminal cysteine;

wherein at least a part of the sequence of HBcAg from between residue 145 (or 147 or 155) and the C-terminal cysteine comprising one or more of the arginine repeats is absent.

Where the protein of the invention comprises both a first epitope from a protein other than HBcAg in place of the absent arginine repeat(s) and a second epitope from a protein other than HBcAg in the e1 loop, the protein typically comprises the following elements linked in an N- to C-terminal direction:

(i) an N-terminal part of the HBcAg sequence comprising e.g. residues 1 to 67 (or 1 to 74 or 1 to 78);

(ii) an epitope from a protein other than HBcAg,

(iii) a second part of the HBcAg sequence comprising e.g. residues 91 to 144 (or 91 to 146, 91 to 154, 86 to 144, 86 to 146, 86 to 154, 80 to 144, 80 to 146 or 80 to 154);

(iv) a further epitope from a protein other than HBcAg, and

(v) a third part of the HBcAg sequence comprising the C-terminal cysteine;

wherein at least a part of the sequence of HBcAg from between residue 145 (or 147 or 155) and the C-terminal cysteine comprising one or more of the arginine repeats is absent

As will be apparent from the above, the inventors specifically contemplate modifying the HBcAg sequence in a number of ways, including deletion of one or more of the arginine repeats, insertion of a heterologous epitope in place of the deleted repeat(s) and insertion of a second heterologous in the e1 loop. However, further modification of the HBcAg sequence is possible. Such further modification may be by way of substitution, insertion, deletion or extension. The size of an insertion, deletion or extension may, for example, be from 1 to 200 aa, from 1 to 100 aa or from 1 to 50 aa, from 1 to 20 aa or from 1 to 6 aa in the sequence of BBcAg. Substitutions may involve a number of amino acids up to, for example, 1, 2, 5, 10, 20 or 50 amino acids over the length of the HBcAg sequence. The modified protein generally retains the ability to form particles. Substitutions will generally be conservative and may be made, for example, according to the following Table, in which amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Each part of the HBcAg sequence in the protein of the invention preferably has at least 70% sequence identity to the corresponding sequence of a natural HBcAg protein, such as the protein having the sequence shown in SEQ ID NO: 2. More preferably, the identity is at least 80%, at least 90%, at least 98%, at least 97% or at least 99%. Methods of measuring protein sequence (and nucleic acid sequence) identity are well known in the art. For example, the UWGCG Package provides the BESTFIT programme (Devereux et al (1984) Nucleic Acids Research 12, p.387-395). Similarly, the PILEUP and BLAST algorithms can be used to line up sequences (for example as described in Altschul S. F. (1993) J. Mol. Evol. 36:290-300 and Altschul, S. F. et al (1990) J. Mol. Biol. 215:403-10).

The protein of the invention may self-assemble into particles which may closely resemble the particles formed by native HBcAg. The particles may be from 20 to 40 nm in diameter, but are preferably about 27 nm in diameter (which is the size of native HBcAg particles). They contain no detectable or reduced amounts of nucleic acid (DNA and RNA) compared to particles of native HBcAg. They may contain from 160 to 260 monomers of the protein of the invenfion, but preferably they contain approximately 180 or approximately 240 monomers (which are the numbers of monomers in native HBcAg particles).

Determination of the particulate nature of a protein according to the invention may be carried out by size exclusion chromatography and/or electron microscopy. Determination of the DNA content of the particles may be carried out by agarose gel electrophoresis or spectrophotometry. A method adapted from Birnbaum and Nasal (1990, J. Virology 64 3319-3330) may be used. The protein may be digested with Proteinase K and the nucleic acid extracted using a commercial DNA recovery kit (e.g. Qiagen, QIAquick™ PCR Purification Kit). Purified DNA may be visualised using a high sensitivity DNA stain (e.g. Novex, SYBER Green I™) in a 1.5% agarose gel, following electrophoresis. The DNA product obtained following extraction may be quantified using the optical density (OD) 260 nm:280 nm ratio according to Sambrook et al. (1989, Molecular cloning—A laboratory manual, second edition, published by Cold Spring Harbor Laboratory Press), for example using a Pharmacia Biotech Ultraspec 2000™.

The Epitopes

As a general rule, epitopes inserted into the protein of the invention should not prevent the folding of HBcAg or its self-assembly into particles. In addition, for improved immunogenicity, B-cell epitopes should be displayed on the surface of the particle. T-cell epitopes do not need to be displayed on the surface of the particle for optimal presentation.

There are three preferred regions for insertion of the epitopes, namely the C-terminus in place of deleted arginine repeat(s), the e1 loop and the N-terinus. These three regions all tolerate well insertion of foreign sequences. When an epitope is placed in the e1 loop of HBcAg, it may be inserted in the sequence of amino acid residues 68 to 90, 69 to 90, 71 to 90, 75 to 85 or 78 to 83. Most preferred is to insert the epitope between residues 79 and 80 or 80 and 81. HBcAg residues from the e1 loop may be deleted in proteins of the invention, so that the inserted epitope may replace all or part of the sequence of the loop.

A heterologous epitope present in a protein of the invention may be a B-cell epitope or a T-cell epitope. In the case that an epitope is a T-cell epitope it may be a T-helper (Th) cell epitope (either a Th1 or Th2 epitope) or a cytotoxic lymphocyte (CTL) epitope.

The protein of the invention may contain more than one heterologous epitope, for example up to 2, 3, 5 or 8 heterologous epitopes, and in this case each epitope may be present in the same site or at different sites in HBcAg. In a preferred embodiment of the invention, one of the epitopes is a T-helper cell epitope and another is a B-cell or a CTL epitope. The presence of the T-helper cell epitope enhances the immune response against the B-cell or CTL epitope. Where there are two or more heterologous epitopes in the protein of the invention, they may be from the same organism or the same protein. Indeed, the epitopes may be the same; this allows a doubling or further multiplication of the number of the epitope presented on the particles.

The size of the sequence comprising an epitope inserted in the protein of the invention can vary between broad limits, but will generally be from 6 to 120 aa, for example from 6 to 80 aa or 6 to 40 aa. The epitope may be conformational or linear.

The choice of epitope depends on the disease that it is wished to vaccinate against. Typically, the epitope is from a pathogen, such as a virus, a bacterium or a protozoan, but it may also be from a cancer associated antigen or an allergen. Examples of pathogens whose epitopes may be inserted include hepatitis A virus (HAV), HBV, hepatitis C virus (HCV), influenza virus, foot-and-mouth disease virus, poliovirus, herpes simplex virus, rabies virus, feline leukemia virus, human immunodeficiency virus type 1 (HIV1), human immunodeficiency virus type 2 (HIV2), simian immunodeficiency virus (SIV), human rhinovirus, dengue virus, yellow fever virus, human papilloma virus, Plasmodium falciparum (a cause of malaria) and bacteria such as Mycobacteria, Bordetella, Salmonella, Escherichia, Vibrio, Haemophilus, Neisseria, Yersinia and Brucella. Specifically, the bacterium may be Mycobacterium tuberculosis—the cause of tuberculosis; Bordetella pertussis or Bordetella parapertussis—causes of whooping cough Salmonella typhimurium—the cause of salmonellosis in several animal species; Salmonella typhi—the cause of human typhoid; Salmonella enteritidis—a cause of food poisoning in humans; Salmonella choleraesuis—a cause of salmonellosis in pigs; Salmonella dublin—a cause of both a systemic and diarrhoeal disease in cattle, especially of new-born calves; Escherichia coli—a cause of food poisoning in humans; Haemophilus influenzae—a cause of meningitis; Neisseria gonorrhoeae—a cause of gonnorrhoeae; Yersinia enterocolitica—the cause of a spectrum of diseases in humans ranging from gastroenteritis to fatal septicemic disease; Brucella abortus—a cause of abortion and infertility in cattle and a condition known as undulant fever in humans; or Clostridium difficile—a cause of pseudomembranous colitis.

Examples of antigens whose epitopes may be inserted are the pre-S1, pre-S2 and S antigens of HBV; the HAV surface antigens; the HCV surface antigens, core protein and NS3 protein; the HIV antigens gp120, gp160, gag, pol, Nef, Tat and Ref; the malaria antigens such as the circumpsorozoite proteins; the influenza antigens HA, NP and NA; the herpes virus antigens EBV gp340, EBV gp85, HSV gB, HSV gD, HSV gH and HSV early protein; the human papilloma virus antigens E4, E6 and E7; the cancer antigens carcinoembryonic antigen (CEA), P53, ras and myc; the pertactin antigen from Bordetella pertussis; and house dust mite allergen.

The invention is particularly suited to prophylactic or therapeutic vaccination against HBV since the carrier protein HBcAg is from HBV, and epitopes from the pre-S1, pre-S2 and S regions of HBV are particularly preferred. A pre-S1, pre-S2 or S insert is typically at least 6 amino acids in length, for example from 6 to 120 aa, 8 to 80 aa or 10 to 40 aa. The insert may include, for example, the residues at pre-S1 positions 1-9, 10-19, 20-29, 30-39, 40-49, 50-59, 60-69, 70-79, 80-89, 90-99, 100-109 or 110-119 or the residues at pre-S2 positi 120-129, 130-139, 140-149, 150-159, 160-169 or 170-174. Particularly preferred fragments are those corresponding to pre-Si residues 20-47 and pre-S2 residues 139-174. Pre-S1 residues 21-28 correspond to a human T-cell epitope. Also preferred are fragments corresponding to S residues 110-147 and 110-157 (counting the first residue of the S sequence as residue 1).

Making the Proteins of the Invention

The proteins of the invention are generally made by recombinant DNA technology. The invention includes a nucleic acid molecule (e.g. DNA or RNA) encoding a protein of the invention, such as an expression vector.

The nucleic acid molecule may encode a protein in which one or more of the arginine repeats has been deleted and replaced with a restriction enzyme site unique to the nucleic acid molecule, such as an XbaI site. The nucleic acid molecule may also contain a unique restriction enzyme site in the sequence encoding the e1 loop and/or in the N-terminus. The unique restriction enzyme sites allow sequences encoding epitopes to be inserted into the nucleic acid molecule, for example in place of the deleted arginine repeat(s) or in the e1 loop.

A protein of the invention may be produced by culturing a host cell containing a nucleic molecule encoding the protein under conditions in which the protein is expressed, and recovering the protein: Suitable host cells include bacteria such as E. coli, yeast, mammalian cell lines and other eukaryotic cell lines, for example insect Sf9 cells.

The vectors constituting nucleic acid molecules according to the invention may be, for example, plasmid or virus vectors. They may contain an origin of replication, a promoter for the expression of the sequence encoding the protein, a regulator of the promoter such as an enhancer, a transcription stop signal, a translation start signal and/or a translation stop signal. The vectors may also contain one or more selectable marker genes, for example an ampicillin resistance gene in the case of a bacterial plasmid or a neomycin resistance gene for a mammalian vector. Vectors may be used in vitro, for example for the production of RNA or used to transform or transfect a host cell. The vector may also be adapted to be used in vivo, for example in a method of gene therapy or DNA vaccination.

Promoters, enhancers and other expression regulation signals may be selected to be compatible with the host cell for which the expression vector is designed. For example, prokaryotic promoters may be used, in particular those such as the trc promoter suitable for use in E. coli strains (such as E. coli HB101). A promoter whose activity is induced in response to a change in the surrounding environment, such as anaerobic conditions, may be used. Preferably an htrA or nirB promoter may be used. These promoters may be used in particular to express the protein in an attenuated bacterium, for example for use as a vaccine. When expression of the protein of the invention is carried out in mammalian cells, either in vitro or in vivo, mammalian promoters may be used. Tissue-specific promoters, for example hepatocyte cell-specific promoters, may also be used. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, herpes simplex virus promoters and adenovirus promoters. All these promoters are readily available in the art.

A protein according to the invention may be purified using conventional techniques for purifying proteins. The protein may, for example, be provided in purified, pure or isolated form. For use in a vaccine, the protein must generally be provided at a high level of purity, for example at a level at which it constitutes more than 80%, more than 90%, more than 95% or more than 98% of the protein in the preparation. However, it may be desirable to mix the protein with other proteins in the fmal vaccine formulation, for example other proteins comprising pre-S1, pre-S2 or S sequence of HBV. The protein is preferably substantially free from nucleic acid (DNA and RNA).

Vaccines

The primary use of the proteins of the invention is as therapeutic or prophylactic vaccines. The invention includes a pharmaceutical composition (e.g. a vaccine composition) comprising a protein of the invention, a particle of the invention or a nucleic acid molecule of the invention and a pharmaceutically acceptable carrier or diluent.

The principle behind prophylactic vaccination is to induce an immune response in a host so as to generate an immunological memory in the host. This means that, when the host is exposed to the virulent pathogen, it mounts an effective protective) immune response, i.e an immune response which inactivates and/or kills the pathogen. The invention could form the basis of a prophylactic vaccine against a range of diseases, such as HBV, HAV, HCV, influenza, foot-and-mouth disease, polio, herpes, rabies, AIDS, dengue fever, yellow fever, malaria, tuberculosis, whooping cough, salmonellosis, typhoid, food poisoning, diarrhoea, meningitis and gonnorrhoeae. The epitopes in the protein of the invention are chosen so as to be appropriate for the disease against which the vaccine is intended to provide protection.

The principle behind therapeutic vaccination is to stimulate the immune system of the host to alleviate or eradicate a disease or condition. There are a number of diseases and conditions which may be susceptible to therapeutic vaccination, such as chronic viral diseases including chronic HBV and chronic HCV, cancer, and allergies such as asthma, atopy, eczema, rhinitis and food allergies.

Chronic viral diseases arise when the immune system of an infected host fails to eliminate the virus, allowing the virus to persist in the host for a long period of time. The invention may be used to induce the immune system of the chronically infected individual so as to eliminate the virus. For example, it is believed that patients with chronic hepatitis have an inadequate T-cell response, and that stimulation of an appropriate T-cell response can eliminate the virus. Thus, in order to treat viral hepatitis using the invention, T-cell epitopes may be inserted into the protein of the invention, such as T-cell epitopes from the pre-S1 and pre-S2 regions of HBV.

Similarly, in the case of cancer, it is believed that enhancement of the T-cell response to tumour antigens may help the immune system to destroy the tumour. It is believed that allergic diseases are caused at least in part by an unbalanced T-cell response in which an inflammatory Th2 responses dominates over an antagonistic Th1 response, and that allergies may therefore be treated by enhancing the Th1 response. This can be achieved according to the invention by using a protein which stimulates a Th1 response.

More than one protein according to the invention may be administered to a patient. Furthermore, a protein according to the invention may be used in combination with one or more other compositions. For example, in the treatment of chronic HBV a protein according to the invention may be used in combination with interferon gamma, Lamivudine™, or another immunotherapeutic agent such as Hepacare™ (formerly known as Hepagene™). The protein according to the invention and the other composition may be administered simultaneously or sequentially.

Suitable carriers and diluents for inclusion in pharmaceutical compositions of the invention are isotonic saline solutions, for example phosphate-buffered saline. The composition will normally include an adjuvant, such as aluminium hydroxide. The composition may be formulated for parenteral, intramuscular, intravenous, intranasal, subcutaneous or transdermal administration. The composition comprises the protein, particles or nucleic acid in a prophylactically or therapeutically effective amount. Typically, the protein or particles are administered at a dose of from 0.01 to 30 μg/kg body weight, preferably from 0.1 to 10 μg/kg, more preferably from 0.1 to 1 μg/kg body weight. The nucleic acid of the invention may be administered directly as a naked nucleic acid construct using techniques known in the art or using vectors known in the art. The amount of nucleic acid administered is typically in the range of from 1 μg to 10 mg, preferably from 100 μg to 1 mg. The vaccine may be given in a single dose schedule or a multiple dose schedule. The routes of administration and doses given above are intended only as a guide, and the route and dose may ultimately be at the discretion of the physician.

### EXPERIMENTAL SECTION

### Experiment 1

1. Materials and Methods

New plasmid constructs were generated by inverse PCR so that three or four C-terminal arginine repeat regions were deleted and a SpeI restriction site was introduced to allow insertion of replacement sequences coding for B and T cell epitopes (FIG. 1).

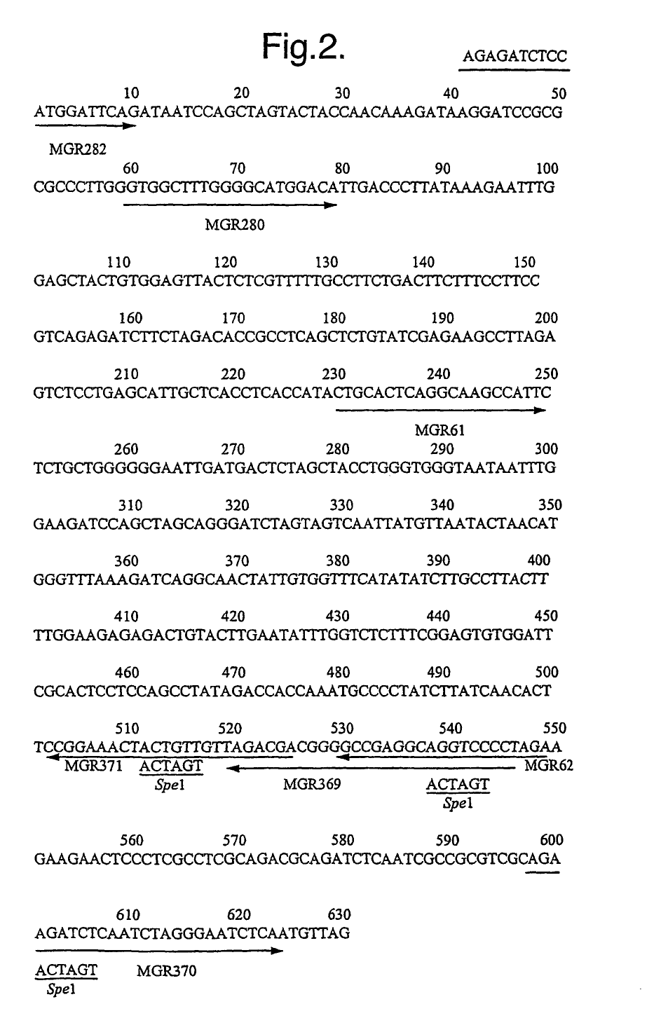

The plasmid templates for the inverse PCR were ptrc/core and ptrc/core-S1 which encode respectively for non-hybrid hepatitis B core and hybrid hepatitis B core containing amino acids 20-47 of the pre-S1 sequence of hepatitis B surface protein inserted between amino acids 79 and 80 of the immunodominant e1 loop. Three oligonucleotide primers (Table 1 and FIG. 2) were used for the PCR reaction. These primers introduce a unique SpeI restriction site in the PCR fragments. The primers were also designed to generate new fragments that were truncated at residues 146 or 154 but maintained 7 residues of the C-terminus including the terminal cysteine at position 185 which is thought to be important for maintaining particle stability by formation of disulphide bonds (FIG. 1).

1.1 Construction of Parental Truncated Plasmids

Using primers MGR371/370 or MGR369/370 (Table 1 and FIG. 2), inverse PCR fragments are generated from plasmid templates of ptrc/core or ptrc/core-S1. This procedure removes 69 nucleotides (encoding for 23 amino acids (aa155-177)) and 93 nucleotides (encoding for 31 amino acids (146-177)) respectively. The PCR fragments sizes were confirmed by analysis on agarose gels and then digested with SpeI restriction endonuclease followed by purification on agarose gels and self-ligation to generate plasmids pTCR₁₄₆, pTCR₁₅₄ and pTCSR₁₄₆ and pTCSR154. pTCR plasmids are derived from the ptrc/core template and pTCSR plasmids are derived from the ptrc/core-S1 templates. The 146 and 154 numbering denotes the amino acid number at the truncation point. The four parental truncated plasmids were used to transform E.coli HB101 cells and positive colonies were tested by diagnostic PCR using oligonucleotide primers MGR61/MGR168. Core protein expression was confirmed by immunoblotting of bacterial cell lysates using a mouse anti-core antibody.

1.2 Subcloning of Replacement Sequences into Truncated Parental Plasmids

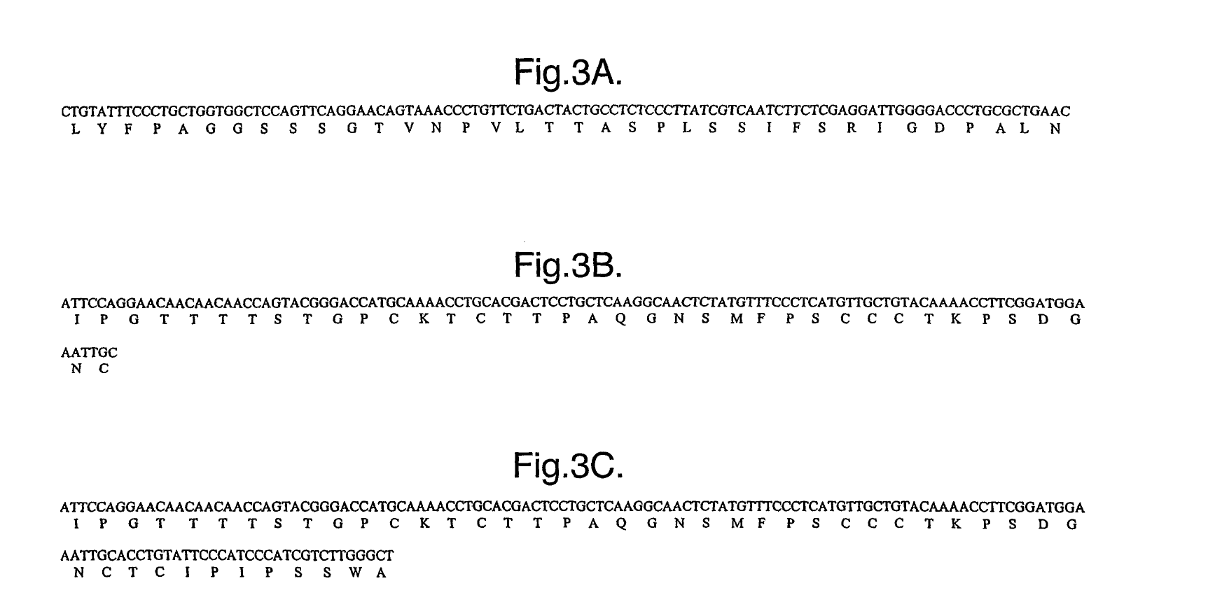

Three sequences have been subcloned into the 3′ end of the truncated parental plasmids described in section 1.1. These include sequences encoding for amino acids 110-147 and 110-157 of the small hepatitis B surface protein, and aa20-55 of the S2 region of the middle hepatitis B surface protein (FIG. 3).

For insertion of the 110-157 sequence (plus 2 amino acids resulting from the NheI restriction site) oligonucleotide primers MR245-247 (Table 1B) were used to generate a PCR fragment of 147 nucleotides using pMBdSRE/17 as template (FIG. 3). This plasmid encodes for the small hepatitis B surface protein (adw subtype) for expression in mammalian cells using the mouse metallothionine promoter.

For insertion of the 110-147 sequence (plus 2 amino acids from the NheI site) oligonucleotide primers MGR247/264 (Table 1B) were used to generate a PCR fragment of 120 nucleotides using pMBdSRE/17 as template (FIG. 3).

For insertion of the 20-55 sequence (plus 2 residues from the NheI site) of pre-S2, oligonucleotide primers MGR243/249 (Table 1B) were used to generate a PCR fragment of 114 nucleotides using pMByS2R/8 as template (FIG. 3). This plasmid encodes for the middle hepatitis B surface protein (ayw subtype) under control of the metallothionine promoter for mammalian cell expression.

The PCR fragments were digested with NheI restriction endonuclease and purified on agarose gels. The purified fragments were then ligated with SpeI digested, phosphatase treated parental plasmids (section 1.1). E.coli HB101 cells were then transformed with the resulting plasmids and positive colonies tested by diagnostic PCR using oligonucleotide primers MGR61/168, immunoblotting with antibodies specific for the insert and partial DNA sequencing of the inserts.

2. Results

2.1 Confirmation of Inverse PCR Fragment Generation

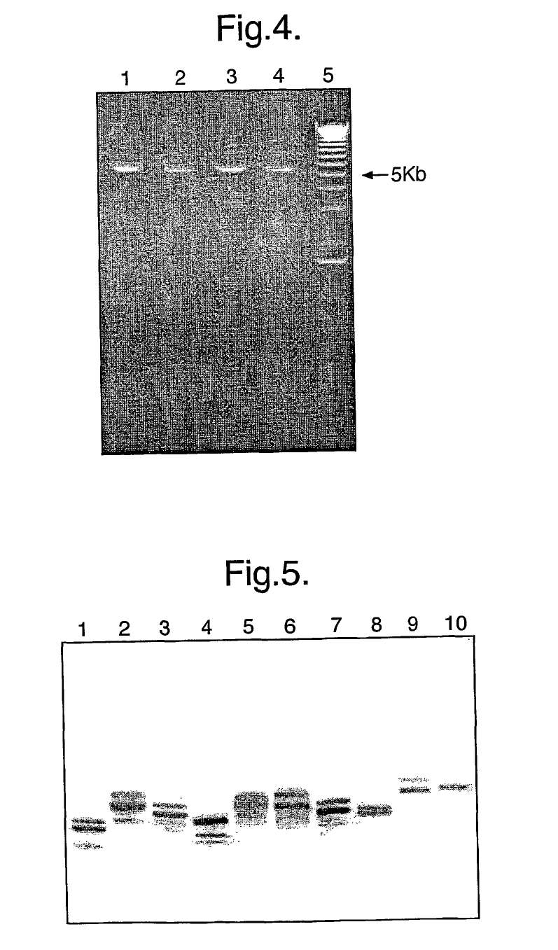

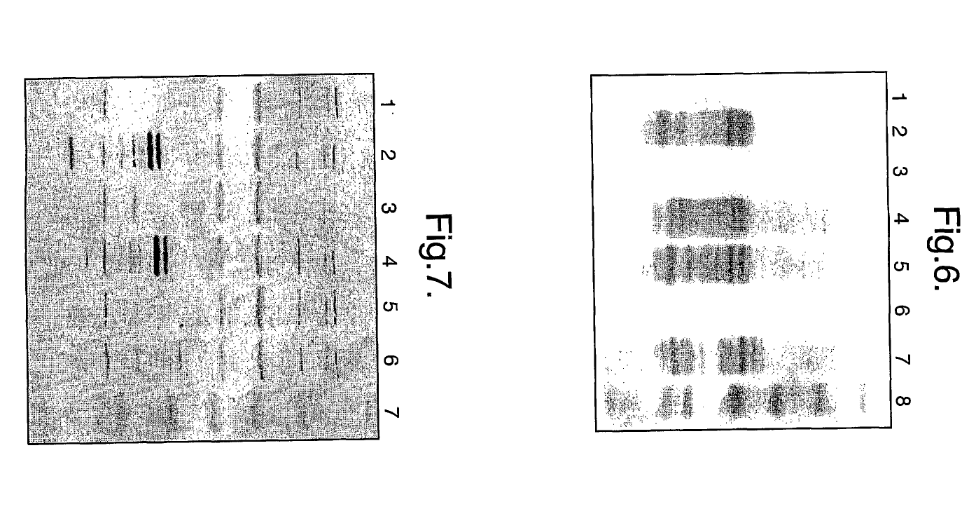

Inverse PCR fragments for pTCR₁₄₆, pTCR₁₅₄, pTCSR₁₄₆ and pTCSR₁₅₄ were analysed by separation on 1% agarose gels (FIG. 4). The PCR fragments were found to be of the appropriate size (approx. 5.2 kb) and were confirmed to be correct by diagnostic PCR (not shown). Immunoblot analysis showed that the parental constructs and those containing the inserted sequences expressed the core protein that was reactive to an anti-core antibody (FIG. 5). Further, confirmation of protein expression of the inserted sequences was shown by immunoblotting using anti-S (FIG. 6) and anti-pre-S2 antibodies (FIG. 7).

Table 1. Oligonucleotide Primers Used for Inverse and Diagnostic PCR

TABLE 1A

| | | |
|---|---|---|
| Oligos | 5′-3′sequence | ptrc/core |

| | | |
|---|---|---|
| MGR61 | CTGCACTCAGGCAAGCCATT | 230bp-249bp |
| MGR62 | GCCGAGGCAGGTCCCCTAGA | 530bp-549bp |
| MGR168 | GAAAATCTTCTCGGATCCGC | from vector |
| | | (pKK233.2) |
| MGR282 | AGAGATCTCCATGGATTCAG | −10bp-10bp |
| MGR280 | GTGGCTTTGGGGCCATGGACA | 60bp-79bp |
| MGR369 | AGGACTAGTGCCTCGGCCCCGTCGTCT | 520bp-546bp |
| MGR370 | AGAACTAGTCAATCTAGGGAATCTCAA | 598bp-624bp |
| MGR371 | TCTTCTAACACTAGTAGTTTCCGG | 502bp-525bp |

Bold denotes SpeI sites

TABLE 1B

| | | |
|---|---|---|
| Oligos | 5′-3′sequence | gene and loca |

| | | |
|---|---|---|
| MGR245 | CAGCTAGCGCAATTTCCATCCGTA | HBsAg 147aa |
| MGR247 | GTTTGTGCTAGCATTCCAGGAACA | HBsAg 110aa |
| MGR264 | CCATAGGTTGCTAGCGAAAGCCCA | HBsAg 157aa |
| MGR243 | TTGCTAGCGTTCAGCGCAGGGTCC | Pre-S2 20aa |
| MGR249 | GTGAGAGCTAGCTATTTCCCTGCT | Pre-S2 55aa |

### Experiment 2

Summary

Full-length and C-terminally truncated hepatitis B core antigen (HBc) derivatives, which carried long foreign amino acid insertions at position 144, were constructed. HBV preS1, preS2, and HIV-1 Gag fragments of 50-100 amino acids in length were used as such insertions, and the appropriate recombinant genes were expressed in E.coli cells. The appropriate chimeric HBc and HBcΔ derivatives were purified and examined antigenically and immunogenically. Subclass analysis of the induced anti-HBC immune response in mice showed that the Ig ratio of IgG1, IgG2a, and IgG2b antibodies was restored from the IgG1>IgG2a≧IgG2b pattern, which is typical for C-terminally truncated HBcΔ derivatives, to IgG2a≧IgG2b≧IgG1, which is typical for full-length HBc derivatives, after immunisation with C-terminally truncated HBcΔ derivatives which carried long C-terminal additions of 50-100 amino acids in length.

Materials and Methods

Bacterial Strains

E.coli strains RR1 (F, hsdS20 (r⁻_{b}, m⁻_{b}), recA⁺, ara-14, proA2, lacY1, galK2, rpsL20 (Sm^{r}), xyl-5, mtl-1, supE44, λ⁻), and K802 (hsdR, gal, met, supE, mcrA, mcrB) were used for selection and expression of chimeric genes, respectively.

Animals

ALB/C (H-2^{d}) female mice were used approximately 7-10 weeks old, weight 20 mg. New Zealand white strain female rabbits were used for obtaining polyclonal antibodies.

Construction of HBc Derivatives

Vectors based on plasmids pHBc3 and pHBc16-15. Vector pHBc3 was constructed by putting the HBc gene underthe control ofthe tandem repeat of E.coli trp promoters. Vector pHBc16-15 was constructed by insertion of an oligonucleotide linker carrying Cla I/Eco RV restriction sites into position 144 of the HBc gene.

Construction ofchimeric HBc derivatives. The structure of the HBc and HBcΔ derivatives is shown in Table 2. The recombinant genes were constructed by insertion of the appropriate HBV preS1, preS2, and HIV-1 gag fragments into the Cla I site ofthe pHBc16-15 vector, with or without in-frame junction to the C-terminal part of the HBc gene.

Purification of Chimeric HBc Derivatives

E.coli cells were grown overnight on a rotary shaker at 37° C. in 750 ml flasks containing 300 ml of M9 minimal medium supplemented with 1% casamino acids (Difco Laboratories, Sparks, USA) and 0.2% glucose. An optical density OD₅₄₀ of 2-5 was usually reached. Generally, cells were pelleted and lysed by 30 min incubation on ice in lysis buffer containing 50 mM Tris-HCl (pH 8.0), 5 nM EDTA, 50 μg/ml PMSF, 2 mg/ml lysozyme and then ultrasonicated 3 times for 15 s at 22 kHz. Lysates were then adjusted to 10 mM MgCl₂, and 20 μg/ml DNAase. After low speed centrifugation, proteins were precipitated from the supernatant with ammonium sulfate at 33% saturation for 1-2 h at 4° C. Pellets were resuspended in a standard PBS buffer containing 0.1% Triton X-100™, and 5 ml of the solutions were loaded on a Sepharose CL4B™ column (2.5×85 cm) and eluted with PBS buffer without Triton X-100. The presence of HBc polypeptides in fractions was tested by PAGE. Positive fractions were pooled and concentrated by ammonium sulfate precipitation at 33% saturation for 20 h at 4° C. Pellets were resuspended in PBS, or in Tris-saline buffer, 10 mM Tris-HCl (pH7.5), 150 mM NaCl, to a final concentration of about 5-20 mg/ml, dialyzed overnight against 2000 volumes of the same buffer and stored at −70° C. or at −20° C. in 50% glycerol.

Polyacrylamide Gel Electrophoresis and Western Blotting

For PAGE analysis, bacteria were pelleted, suspended in SDS-gel electrophoresis sample buffer containing 2% SDS and 2% 2-mercaptoethanol and lysed by heating at 100° C. for 5 min. The proteins were separated by Laemmli's polyacrylamide gel electrophoresis (PAGE) in a slab gel (150×150×0.75 mn) apparatus with a gradient 12-18% running gel and a 4% stacking gel. Western blotting was performed in general as described by Towbin et al (1979) in Proc. Natl. Acad. Sci. USA 76 4350-4354. Nitrocellulose sheets (0.2μ, Millipore, Bedford, USA) were incubated with anti-HBc antibodies and anti-preS1 antibody in dilutions of 1:100 to 1:1000 overnight and then with anti-mouse IgG peroxidase conjugate (1:1000) for 1-2 h at room temperature. The reaction was developed with 3,3′-diaminobenzidine. In parallel, gels were silver-stained according to Ohsawa and Ebata (1983) Anal. Biochem. 135 409-415.

Inmunisations

Mice (five per group) were immunised at day 0 intraperitoneally with 0.02 mg of chimeric 20 particles in complete Freund's adjuvant (CFA, Difco) followed by two booster immunisations in Freund's incomplete adjuvant (IFA, Difco) given at days 10 (0.01 mg intraperitoneally) and 24 (0.01 mg intraperitoneally and 0.01 mg subcutaneously). Sera obtained on day 32 were analysed by ELISA for reactivity with HBc particles.

ELISA

For the ELISA, recombinant HBc particles were coated onto 96-well microtiter plates by air-drying in a chemical hood overnight. Wells were blocked with 0.5% BSA in PBS for 1 h, incubated with serial dilutions ofthe various antibodies for 1 h at 37° C. and processed with the appropriate second antibodies conjugated to horse radish peroxidase (Sigma) according to the protocols of the manufacturers. Plates were washed 5 times between incubations with 0.05% Tween-20™ in PBS, and 5 times with distilled water to remove Tween-20. Optical absorbances were measured at 492 nm in an automatic Immunoscan MS™ reader. The titres were calculated as the negative logarithms of the EC50 (effective concentration, 50%) seru dilution on the basis of signoidal dose-response curves. GraphPad Prism® version 3.02 software was used in the mean titre calculations.

Results

Imunogenicity of Recombinant Proteins. To measure the immunogenicity of HBc carrier and inserted preS1, preS2, and Gag sequences, individual mice sera were repeatedly tested by direct ELISA using recombinant HBcAg and synthetic preS1, preS2, and HIV-1 p24 peptides on solid support. Immunisation with chimeric particles induced high levels of anti-HBc and relatively low levels of anti-insertion antibodies (not shown).

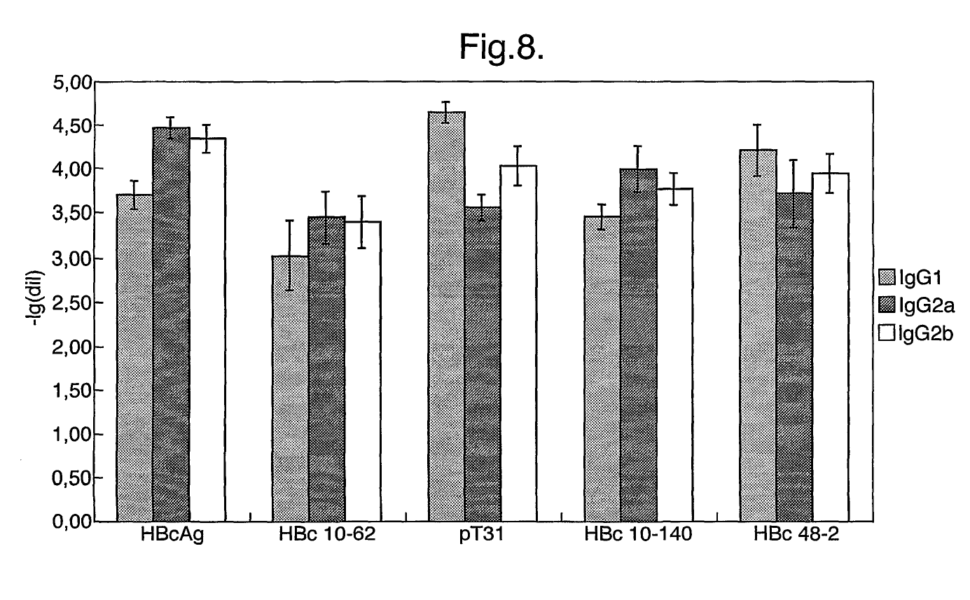

Induction of Different Immunoglobulin Subclasses by Chimeric HBcΔ-preS1 (20-47) Particles In order to average obtained immunisation data and to make them more informative for comparative subclass analysis of induced immunoglobulins, we calculatedmeantitres for each group of immunised animals as the negative logarithms of the EC50 (effective concentration, 50%) serum dilution on the basis of sigmoidal dose-response curves (GraphPad Prism® version 3.02). These data on the anti-HBc response of immunised mice, which allow direct comparison of averaged titres, are given in FIG. 8.

The data presented in FIG. 8 show that the wild type HBcAg induces anti-HBc response with the immunoglobulin subclass distribution IgG2a≧IgG2b>IgG1, whilst the immune response to the C-terminally truncated HBcΔ structure T31 presents the IgG1>IgG2b≧IgG2a subclass distribution pattern. The full-length HBc derivative 10-62, which carries a 50 aa long preS1 insertion, shows a subclass distribution analogous to that of the fill-length HBc vector. Moreover, replacement of the C-terminus of the HBc molecule by a long foreign insertion (50 amino acids of the preS1 sequence) in the HBc derivative 10-140 makes the subclass distribution of the anti-HBc antibodies rather similar to that induced by the full-length HBc structure (FIG. 8). The HBcΔ derivative 48-2 with a 100 aa long insertion of HIV-1 Gag occupies an intermediate position in this sense between wild type HBcAg and C-terminally truncated HBcΔ T31 structures.

Table 2. Structure of HBc derivatives with C-terminal insertions. Amino acids appearing at the HBc and insertion sequence junctions are shown in lowercase.

Full-Length HBc Derivatives

| | | |
|---|---|---|
| Construct | Insertion | Sequence |

| | | |
|---|---|---|
| HBc | preS | HBc preS1 HBc |
| 10-62 | 131- | 144 31 40 50 60 70 80 145 |
| | 80 | P krsiskrsis DPAFRANTANPDWDFNPNKDTWPDANKVGAGAPGLGFTPPHGGLLGWSPQ s E . . . |
| HBc9-87 | preS | HBc preS2 HBc |
| 87 | 21- | 144 1 10 20 30 40 50 145 |
| | 54 | P krsi QAMQWNATTFHQTLQDPRVRGLYFPAGGSSSGTVNPVPTVVSPISSIFSRIGDPAL ks E . . . |

C-Terminal Truncated HBc Derivatives

| | | |
|---|---|---|
| Construct | Insertion | Sequence |

| | | |
|---|---|---|
| HBcΔ | preS | Hbc preS1 |
| 10-140 | 131- | 144 31 40 50 60 70 79 |
| | 79 | P krsiskrsis DPAFRANTANPDWDFNPNKDTWPDANKVGAGAFGLGFTPPHGGLLGWSP hdigdycc |
| HBcΔ | preS | HBc preS2 |
| 9-142 | 21- | 144 1 10 20 30 40 50 55 |
| | 55 | P krsi QAMQWNSTTFHQTLQDPRVRGLYFPAGGSSSGTVNPVPTTVSPISSIFSRIGDPALN gdycc |
| HBcΔ | HIV | 144 p17 p24 1 55 |
| 48-2 | p55 | P ns DTGHSSQVSQNYPIVQNIQGQMVHQAISPRTLNAWVKVVEEKAFSPEVIPMFSALSEGATPQDLNTM |
| | 121- | 56 78 |
| | 210 | LNTVGGHQAAMQMLKETINEEAA agmgasla |

1. A protein comprising hepatitis B core antigen (HBcAg) wherein one or more of th four arginine repeats is absent and a C-terminal cysteine residue is present.

2. A protein according to claim 1 wherein a first epitope from a protein other than HBcAg is present in place of the absent arginine repeat(s).

3. A protein according to claim 1 or 2 wherein the first arginine repeat is present and the second to fourth arginine repeats are absent.

4. A protein according to any one of the preceding claims wherein a sequence lying between residues 145 and 182 of HBcAg is absent.

5. A protein according to any one of the preceding claims wherein a sequence lying between residues 150 and 177 of HBcAg is absent.

6. A protein according to any one of the preceding claims which comprises a second epitope from a protein other than HBcAg, the second epitope being in the e1 loop.

7. A protein according to claim 6 wherein the second epitope is a B-cell epitope.

8. A protein according to any one of claims 2 to 7 wherein the first epitope is a T-cell epitope.

9. A protein according to claim 8 wherein the first epitope is a T-helper cell epitope and the second epitope is a B-cell epitope.

10. A protein according to claim 6 which comprises said first and second epitopes wherein the epitopes are the same.

11. A protein according to any one of claims 2 to 10 wherein the first and/or the second epitope is from hepatitis B virus (HBV).

12. A protein according to claim 11 wherein the first and/or the second epitope is from the pre-S1, pre-S2 or S region of HBV.

13. A protein according to claim 1 comprising the following elements linked in an N-terminal to C-terminal direction:
(i) an N-terminal part of HBcAg which mediates the formation of particles, and
(ii) a C-terminal part of HBcAg comprising the C-terminal cysteine;
wherein at least a part of the sequence of HBcAg from between said N-terminal part and said C-terninal part comprising one or more of the arginine repeats is absent.

14. A protein according to claim 1 comprising the following elements linked in an N-to C-terminal direction:
(i) an N-terminal part of HBcAg which mediates the formation of particles,
(ii) an epitope from a protein other than HBcAg, and
(iii) a C-terminal part of HBcAg comprising the C-terminal cysteine;
wherein at least a part of the sequence of HBcAg between said N-terminal part and said C-terminal part comprising one or more of the arginine repeats is absent and is replaced by said epitope.

15. A protein according to claim 1 comprising the following elements linked in an N-to C-terminal direction:
(i) an N-terminal part of the HBcAg sequence comprising residues 1 to 67,
(ii) an epitope from a protein other than HBcAg,
(iii) a second part of the HBcAg sequence comprising residues 91 to 144, and
(iv) a third part of the HBcAg sequence comprising the C-terminal cysteine;
wherein at least a part of the sequence of HBcAg from between residue 145 and the C-terminal cysteine comprising one or more of the arginine repeats is absent.

16. A protein according to claim 1 comprising the following elements linked in an N-to C-terminal direction:
(i) an N-terminal part of the HBcAg sequence comprising residues 1 to 67;
(ii) an epitope from a protein other than HBcAg,
(iii) a second part of the HBcAg sequence comprising residues 91 to 144;
(iv) a further epitope from a protein other than HBcAg;
(v) a third part of the HBcAg sequence comprising the C-terninal cysteine;
wherein at least a part of the sequence of HBcAg from between residue 145 and the C-terminal cysteine comprising one or more of the arginine repeats is absent.

17. A particle comprising multiple copies of a protein as claimed in any one of the preceding claims.

18. A nucleic acid molecule encoding a protein as claimed in any one of claims 1 to 16.

19. A nucleic acid molecule according to claim 18 which is an expression vector.

20. A host cell transformed or transfected with a nucleic acid molecule as claimed in claim 18 or 19.

21. A process for producing a protein as claimed in any one of claims 1 to 16, which process comprises culturing a host cell containing a nucleic acid molecule which encodes the protein under conditions in which the protein is expressed, and recovering the protein.

22. A nucleic acid molecule encoding a protein as claimed in claim 1 wherein the sequence encoding one or more of the four arginine repeats of HBcAg is deleted and replaced with a restriction enzyme site unique to the nucleic acid molecule.

23. A pharmaceutical composition comprising a protein as claimed in any one of claims 1 to 16, a particle as claimed in claim 17 or a nucleic acid molecule as claimed in claim 18 or 19 and a pharmaceutically acceptable carrier or diluent.

24. A protein according to any one of claims 1 to 16, a particle according to claim 17 a nucleic acid molecule according to claim 18 or 19 for use in a method of prophylactic or therapeutic vaccination of the human or animal body.

25. A protein, particle or nucleic acid molecule according to claim 24 for use in a method of prophylactic or therapeutic vaccination of the human or animal body against HBV.

26. Use of a protein according to any one of claims 1 to 16, a particle according to claim 17 or a nucleic acid molecule according to claim 18 or 19 for the manufacture of a medicament for prophylactic or therapeutic vaccination of the human or animal body against HBV.

27. A method of vaccination or therapy of a subject, which method comprises administering to the subject a protein as claimed in any one of claims 1 to 16, a particle as claimed in claim 17 or a nucleic acid molecule as claimed in claimed 18 or 19.
